(19) United States
(12) Patent Application Publication
(10) Pub. No.: US 2006/0246444 A1
(43) Pub. Date: Nov. 2, 2006

(54) Process control based on analysis of microbial populations

(76) Inventors: Josephus Mauritius Bernardus Maria van der Vossen, Zeist (NL); Frank Henri Johan Schuren, Veenendaal (NL); Christiaan Montijn, Amsterdam (NL)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/538,532

(22) PCT Filed: Dec. 12, 2003
(86) PCT No.: PCT/NL03/00885

§ 371 Date: Mar. 24, 2006

(30) Foreign Application Priority Data
Dec. 12, 2002 (NL) 1022152

(51) Int. Cl. C12Q 1/68; C12Q 1/04

(52) U.S. Cl. 435/6; 435/34

(57) ABSTRACT

The present invention relates to determining environmental conditions for the purpose of process control by means of microorganisms. The present invention makes use of the principle that changes in the composition of a microbial population after applying an external stimulus are characteristic of the nature of the stimulus received by the microbial population. By measuring these changes, the composition of the microbial population can be used as a specific sensor to determine the physical chemical environmental conditions, to determine the extent of change in this environmental condition, to determine the nature and intensity of the stimulus, and to be able to measure the effects of different stimuli on various processes and thus control these processes.

The present invention relates to a method for determining an environmental condition. In particular, the invention relates to a method for determining an environmental condition for the purpose of process control.

Of many industrial and natural processes, such as the fermentative preparation of food or the action of the human digestive tract, the process parameters and the prevailing process conditions cannot easily be determined. As a result of this absence of knowledge concerning the prevailing process conditions, intervention therein or control thereof is very difficult and mostly inefficient. The condition of a process is influenced to a large extent by the environment. However, these environmental conditions are also often difficult to determine. A method for determining an environmental condition can therefore provide valuable information about the prevailing influences on a particular process. Also, information about the prevailing process conditions would considerably facilitate the control of this process.

In this context, the term environmental condition is meant to refer to the physical and/or chemical condition of the environment in a broad sense.

The environmental condition in processes such as biofilm formation, mineralization in the soil, tooth decay, water purification, biodegradation, product quality loss and infection can, for instance, be derived from physical or chemical parameters which play a role in that process.

Nowadays, a large variety of environmental conditions can easily be measured using instruments specially developed for this purpose. Also, specific biosensors for measuring a number of chemical compounds are available. However, most chemical compounds cannot be measured easily or cannot be measured at all.

Controlling or monitoring a physical environmental condition such as (barometric) pressure, temperature, light regime, noise, radiation, moisture content and/or atmospheric humidity, a chemical environmental condition such as the concentration of a chemical component or chemical substance and, for instance, acidity, is now highly desired in many industries.

A problem in the determination of changes in above-mentioned environmental conditions is that, in many cases, they involve very small changes which alone can influence a process to a great extent. However, by the current means and methods, such very small changes in the environment are hardly measurable, if at all.

A further problem of the determination of changes in above-mentioned environmental conditions is that it is not always known which physical or chemical parameters of the environment play a role in a particular process or exert an influence thereon. In that case, the changes in the environmental condition cannot be specified or are difficult to specify, which complicates the control of the process taking place in that environment.

Microorganisms are very sensitive to the condition of their environment. In fact, the condition of a microorganism reflects the condition of its environment. In situations in which one or only a few species are (dominantly) present, it is possible to determine reactions of these microorganisms to the environment by measuring (a part of) the biochemical composition of these microorganisms. In this context, as a biochemical composition, very suitably, (a part of) the composition of the transcriptome, the proteome or the metabolome can be determined because this part of the cellular or biochemical composition of microorganisms reacts very strongly to the environment.

However, in many cases, a population is involved of several to very many species and/or strains or even phyla of microorganisms which are found in different proportions to one another and which often all react to the environmental condition in a different manner. This complicates the measurement of the biochemical composition of a microorganism. On which microorganism should measurements be performed, and which microorganisms do the measurement data obtained come from?

Now it has surprisingly been found that the composition of a microbial population can provide information about prevailing environmental conditions and about prevailing process conditions and also about changes therein. A microbial population as a whole may be used as a measuring instrument for determining an environmental condition and/or a change therein because the composition of this population reflects the prevailing environmental condition.

The present invention provides a method for determining an environmental condition by measuring a composition of a microbial population which is exposed to this environmental condition.

The present invention further provides a method for determining changes in an environmental condition by measuring changes in a composition of a microbial population which is exposed to these changes in an environmental condition.

The present invention further provides a method for determining an environmental condition, comprising measuring a composition of a microbial population exposed to this environmental condition, correlating this composition to a previously compiled data file of a plurality of compositions obtained by exposing this microbial population to a plurality of environmental conditions and determining this environmental condition on the basis of the outcome of this correlation.

By the improved method, processes can be controlled on the basis of the changes in a composition of a microbial population which may or may not be already present in a process or in a process) environment, or is introduced therein with the purpose of determining an environmental condition by means of a method according to the present invention.

The improved method has the advantage that very small changes in the environment, or very low concentrations of the substances to be detected, which could formerly be measured with difficulty or not at all, can now be measured as well.

It is an advantage of the present invention that the speed of the measurement is, at least potentially, high. It is a further advantage of the present invention that analysis results can be stored in a database or data file so that a new measurement can be compared to previously found results so that, in time, the method gets a predictive value. The improved method thus becomes self-learning.

Another advantage of the present invention is that, by the improved method, deviations and changes in an environmental or process condition can already be detected before other, more crucial characteristics of this environmental or process condition demonstrably change. The improved method can thus serve as a warning system.

Still another advantage of the method according to the present invention is that a low environmental condition or stimulus not measurable in itself can be reflected in an intensified manner in a great change of the population composition.

The present invention uses the principle that a microorganism reacts strongly to outside influences. The intrinsic changes in the microorganism after applying an external stimulus (an environmental condition) consist in changes in amounts and nature of biomolecules such as RNA, protein and metabolites. The sum total of such changes is characteristic of the nature of the external stimuli that the microorganism receives.

However, under the influence of a specific stimulus or environmental condition, not only the concentrations of above-mentioned biomolecules in the microorganisms change. As a result of the shifting of optimal environmental conditions and the subsequent intrinsic changes in the microorganism, the growth and competitive capacity of the individual microorganisms in a population also change. This causes the composition of the population to change. An organism which was dominantly present at first may, in this manner, be wholly displaced by an organism which thrives better under the given environmental conditions.

In this manner, a composition of a microbial population may be used as a specific sensor to determine environmental conditions, to determine the extent of change in this environmental condition, to determine the nature and intensity of the stimulus and to be able to measure the effects of different stimuli on various processes and thus control these processes.

A number of methods which measure on populations of bacteria are already known in the literature. The European patent application 1 215 285, for instance, describes the possibility to monitor the feeling of an individual about his own health condition on the basis of his bacterial intestinal flora. Such a method could be used in health examination to thus detect small changes in the health condition which cannot be determined by blood examination. However, measuring a feeling of good or poor health is not the subject of the present application.

The International application WO 97/05282 describes a method for determining the composition of a microbial population by means of in situ hybridization. The publication describes that changes in populations caused by chemical substances can be measured. However, this provides no decisive answer about changes in the environment.

A number of publications are known in which not the composition, but other properties of a population are measured. The German patent application DE 195 43 993 describes the measurement of the density of a microbial population found in the neighborhood of the hazardous substance. The International patent application WO 99/09202 describes a biosensor on the basis of the morphological changes or reporter gene expression in Dictyostelium cells. The German patent application DE 199 17 955 describes an apparatus where the biological oxygen consumption of a mixed population of microorganisms in a flow cell is measured. The U.S. Pat. No. 5,055,397 describes a method in which the survival of soil bacteria is determined. The International patent application WO 97/13873 describes a method in which the microbial growth is investigated. The U.S. Pat. No. 5,563,043 describes a method in which the rate at which the number of living cells decreases is determined. The Japanese patent abstract JP 2001 231598 describes a method in which the decrease in ATP in bacterial cells is determined. The British patent 1 437 458 describes a method in which the metabolic activity of microorganisms is measured by measuring, for instance, cell increase, carbon dioxide production, acidity change or oxygen consumption. However, none of these methods determines the composition of the microbial population according to the present invention.

In the present invention, a composition of a microbial population is understood to mean a group structure of a community of microorganisms involving a community of at least two different groups and which group structure is in a substantially stable condition in a substantially stable environmental condition.

Herein, a group of microorganisms is understood to mean a group of microorganisms which can be distinguished from another group on the basis of one or more specific genotypic or phenotypic characteristics. Such a group may comprise a taxonomic group such as a phylum, a family, a species or a strain, but also a group which is methodologically classified such as a phylogenetic cluster, a ribotype, an isolate, a serotype, or a morphotype.

In the determination of a composition of a microbial population, the ratio in which the different groups are found in the population may be determined, but also the proportion of a group in the population. A ratio or proportion may, for instance, be expressed in a cell number, but also in a weight of a cell or a cell component, such as a weight of a nucleic acid, or a fluorescence intensity. A skilled person will understand that the manner in which a composition of a microbial population is expressed depends on the method by which this composition is determined.

In the present invention, a change in a composition of a microbial population is understood to mean an absolute or relative increase or decrease of the proportion of a group in the population.

Microbial populations which may be used in a method according to the present invention may comprise microorganisms such as bacteria, archaea, fungi, yeasts, protozoa and algae. Preferably, microbial populations of bacteria, yeasts and/or fungi are used in a method according to the invention, most preferably bacteria.

In a method according to the invention, microbial populations introduced into or occurring naturally in a specific process may be used for determining an environmental condition of that same process or a completely different process, preferably, however, the intrinsically present microbial population is used. So, in the control of processes in which specific populations of microorganisms play a role, preferably, the specific populations of microorganisms from these processes will be used.

If a process takes place in the absence of a population of microorganisms, a population of microorganisms can be exposed to the process conditions for a shorter or longer period, for instance by introducing it into the processing room preferably from the start of the process, or by bringing (a part of) the process matter into contact with a population of microorganisms outside the processing room.

If required, the microorganisms can be grown in advance, as, for instance, is done in a process in which a mixed starter culture is used as a graft. In certain embodiments, it is possible to use the population of microorganisms in a composition which is uniformized or standardized or defined in advance. This can, for instance, be the case if the population of microorganisms is exposed to the above-described process conditions outside the processing room, for instance by exposing it to (a part of) the process matter, such as a sample of water, air, food, or when the microorganisms are exposed to certain process conditions for a shorter or longer period for the purpose of determining the process conditions.

In this context, a uniformized or standardized or defined composition is understood to mean a population of microorganisms of which the composition is known under given environmental or process conditions. If such a composition of the population can be obtained repeatedly, for instance by mixing different microorganisms in a known proportion, it can also be considered to be a standard.

It is not required, however, that the microbial population is used in a uniformized or standardized or defined composition. In fact, measuring the population composition of microorganisms used in a method according to the present invention, or a change in this composition, is sufficient to determine the conditions of the environment, or changes therein, as long as the (change in) the population composition of microorganisms can be related to a specific environmental or process condition.

In the present invention, a microbial population composition can comprise a population of microorganisms of which the taxonomic position of the individual members is known. But the use of microbial populations of which the taxonomic position of the individual organisms is unknown is also possible.

As set forth hereinabove, in the present invention, a composition of a microbial population can be determined by carrying out measurements on biomolecules of microorganisms, by means of which the occurrence of different groups, for instance species, of microorganisms in the population can be determined.

Suitable biomolecules which can be measured for determining the occurrence of different groups of microorganisms comprise inter alia biological macromolecules such as polysaccharides, lipids, polypeptides and polynucleotides.

Thus, measurements can be used by which the different taxonomic groups can be identified and optionally quantified, but this is not required.

In particular embodiments, it is therefore possible to determine the composition of a microbial population without determining the taxonomic position of the individual groups in a population or identifying the individual microorganisms.

In the case that such an identification or determination of the taxonomic position does not take place, it is possible to still distinguish the individual microorganisms in a composition of a microbial population on the basis of a “community fingerprint” or biomolecular population pattern. For this purpose, a separation of biological macromolecules occurring in bulk in the microbial population can be used. By such analyses of bulk macromolecules, initially, the individual taxonomic levels in a population cannot be determined and the individual members of the population cannot be identified. However, they can be used to obtain a “community fingerprint” of the population composition. By comparing two of such “community fingerprints”, taken at different times, a change in the population composition can be determined.

Such analyses of bulk macromolecules of microorganisms comprise, for instance, one or two-dimensional electrophoresis (SDS-PAGE) of proteins from the whole cell (Vauterin, L., Swings, J., and K. Kersters. 1994. Handbook of a New Bacterial Systematics. Goodfellow and O'Donnell, eds. San Diego, Calif., Academic Press). Further, for obtaining a “community fingerprint”, nucleic acids from the whole cells of the population can be analyzed by methods such as “genome profiling” (Nishigaki et al. 1991. Chem. Lett. 1097-1100), “bacterial restriction enzyme nucleic acid digest analysis” (BRENDA; Robinson et al. 1982. J. Med. Microbiol. 15:331-8), “pulsed-field gel electrophoresis” (PFGE; Swain et al. 1996. Appl. Environ. Microbiol. 62:994-7) in combination with restriction endonuclease digestion of chromosomal DNA, “(terminal) restriction fragment length polymorphism” ((T)RFLP; Dunbar et al. 2000. Appl. Environ. Microbiol. 66:2943-50; Park et al. 2002. Water Sci. Technol. 46:273-80) including Ribotyping and AFLP™ (Vos et al. 1995. Nucleic Acids Res. 23:4407-14), “amplified ribosomal DNA restriction analysis” (ARDRA; Vaneechoutte et al. 1992. FEMS Microbiol. Lett. 72:227-33), “random amplified polymorphic DNA” (RAPD; Okamura et al. 1993. Proc. R. Soc. Lond. B. Biol. Sci. 253:147-54), “denaturing gradient gel-electrophoresis” (DGGE; Muyzer et al. 1993. Appl. Environ. Microbiol. 59:695-700), “temperature gradient gel electrophoresis” (TGGE; Felske et al. 1997. Microbiology 143:2983-9), “single strand conformational polymorphism” (SSCP; Widjojoatmodjo et al. 1995. J. Clin. Microbiol. 33: 2601-2606.), “plasmid profiling” (Tannock et al. 1990. J. Clin. Microbiol. 28:1225-8), “plasmid fingerprinting” (Tenover. 1985. Clin. Lab. Med. 5:413-36) and/or “shot-gun cloning and sequencing” of amplified nucleic acid fragments (e.g. Giovannoni et al. 1990. Nature 345:60-3) can be used. For an overview of possible methods which can be used in a measurement for determining a composition of a microbial population, reference is made herein to Molecular Microbial Ecology Manual. 1998. Akkermans, van Elsas and de Bruijn, eds. Kluwer Academic Publishers, Dordrecht, the Netherlands, ISBN 0-7923-5343-9, and periodic updates thereof.

Preferably, for determining a composition of a microbial population, measurements are used in which, also, taxonomic identification takes place. For instance, lower taxonomic levels such as strains, varieties, subspecies and species, but also higher taxonomic levels such as genera, families, orders, classes and the like can be determined. It is also possible to determine combinations of different taxonomic levels of microorganisms in a method according to the invention by which a population composition is measured.

For this purpose, marker molecules can very suitably be used for identifying taxonomic groups of microorganisms. Such marker molecules comprise inter alia (parts of) biological macromolecules such as polysaccharides, lipids, polypeptides and polynucleotides which are present in the microorganisms and which are specific to their taxonomic position.

Methods for identifying polypeptides or proteins which can be used as taxon-specific markers for taxonomic identification of microorganisms are known to a skilled person and comprise inter alia a combination of 2D gel electrophoresis and mass spectrometry (MS) and “peptide display library” technology (Smith & Scott. 1993. Meth. Enzymol. 217:228-257.)

Methods for identifying polynucleotides or nucleic acid sequences which are present or can be expressed in one cell population, but not in another, and can therefore be used as taxon-specific marker, are also known to a skilled person and comprise inter alia “subtractive cloning” (Sagerstrom et al. 1997. Annu. Rev. Biochem. 66:751-83), RAPD (Hadrys et al. 1992. Mol. Ecol. 1:55-63) and/or “subtractive hybridization” (el-Adhami et al. 1997. J. Med. Microbiol. 46:987-97).

Further, for identification of taxon-specific markers, the above-described methods for determining a “community fingerprint” can be combined with sequence analysis of nucleic acid fragments isolated and selected from such a pattern. Such a combined method for identifying taxon-specific markers, by combination of bulk and taxon-specific methods, may, for instance, comprise amplification and shot-gun cloning of ribosomal RNA genes from the total population followed by partial sequence analysis of the cloned genes (see e.g. Wilson & Blitchington. 1996. Appl. Environ. Microbiol. 62:2273-8), but also, for instance, separation of bulk-amplified RNA genes by means of TGGE and partial sequence analysis of selected, separated fragments (see e.g. Muyzer. 1999. Curr. Opin. Microbiol. 2:317-22) or, for instance, 2D gel electrophoretic separation of proteins and the manufacture of antibodies against isolated separated proteins.

Suitable taxon-specific polynucleotide marker molecules may comprise, for instance, (fragments of) ribosomal RNA (for instance 5S, 5,8S, 9S, 12S, 16S, 18S, 23S of 25S, or the spacer regions between them), transfer RNA, genomic DNA, plasmid DNA or mitochondrial DNA. Preferably, genomic DNA-related and/or rRNA-related markers are used.

Suitable taxon-specific polypeptide marker molecules may, for instance, comprise (fragments of) intracellular and/or membrane-bound enzymes such as cytochrome b, cytochrome c oxidase, NADH dehydrogenase, ATP synthase and/or esterase. Such polypeptides are so suitable because taxonomically annotated databases are available of them. Also, taxon-specific antigens can be measured in a method according to the invention.

A method according to the invention preferably comprises the use of taxon-specific polypeptide marker molecules such as nucleic acid markers.

For determining a composition of a microbial population by means of taxon-specific markers, use can be made of one or more techniques such as:

in situ measurement techniques such as nucleic acid probe techniques (see inter alia Amann et al. 1990. J. Bacteriol. 172:762-70) and/or immunological measurement techniques; here, different cellular components can be measured separately, without actual separation of them taking place, by using specific or aspecific detection techniques, whose suitability depends on the component to be detected;

electrophoretic and/or chromatographic measurement techniques such as inter alia DGGE; here, the cellular components can be separated on the basis of inter alia size, weight, charge, sensitivity to denaturation, after which detection takes place by means of specific or aspecific detection techniques, whose use depends on the component to be detected (see e.g. Molecular Microbial Ecology Manual. 1998. Akkermans, van Elsas and de Bruijn, eds. Kluwer Academic Publishers, Dordrecht, the Netherlands);

microarray and/or biochip techniques; here, biochemical components are separated on the basis of affinity for a binding partner immobilized on a carrier and detection takes place by means of specific or aspecific detection techniques, whose suitability depends on the component to be detected.

Suitable detection techniques which may be used in relation to above techniques are inter alia autoradiographic detection techniques, detection techniques based on fluorescence, luminescence or phosphorescence and chromogenic detection techniques. These techniques are known in the field of the detection of biomolecules.

Preferably, in the present invention, a composition of a microbial population is measured by using techniques in which specific biomolecules are bound to microarrays of specific binding partners. For instance, microarrays of nucleic acid probes or nucleic acid imitating probes, such as PNA, can be used for detection of taxon-specific polynucleotide markers, or microarrays of binding partners of peptides (“protein chips”) or of antibodies can be used for detection of taxon-specific polypeptide markers.

Methods for developing nucleic acid probes and for working with polynucleotides which can be used in the present invention are known to a skilled person (see inter alia Stahl & Amann 1991. In: Nucleic Acid Techniques in Bacterial Systematics, Stackebrandt and Goodfellow, eds. pp. 205-48 Whiley, Chichester) and have been described in a large number of handbooks including Molecular Cloning: A Laboratory Manual, 2nd Ed., Vol. 1-3, eds. Sambrook et al. Cold Spring Harbor Laboratory Press (1989) and Current Protocols in Molecular Biology, eds. Ausubel et al., Greene Publishing and Wiley-Interscience: New York (1987) and periodic updates thereof.

Methods for developing peptide probes and for working with microarrays for detection of taxon-specific polypeptides which can be used in the present invention are known to a skilled person and have been described in a large number of handbooks including Proteomics, Palzkill ed. Kluwer Academic Publishers, 2002 and Peptide Arrays on Membrane Supports: Synthesis and Applications, Mahler ed. Springer-Verlag New York, 2002.

In a method according to the present invention, use of a DNA microarray is preferred. Such arrays comprise oligonucleotides with sequences which are specific to taxon-specific nucleic acid markers and are, in the present invention, also referred to as oligonucleotide array, solid carrier nucleic acid microarray, DNA array or DNA biochip.

The manufacture of a DNA microarray according to the invention can be carried out by means of methods known to a skilled person. The manufacture and the use of DNA microarrays for the detection of specific nucleic acid sequences have often been described in publications (U.S. Pat. No. 5,571,639; Sapolsky et al., 1999, Genet. Anal.-Biomolecular Eng. 14, 187-192; Chee et al., 1996, Science 274:610-614, Shena et al., 1995, Science 270, 467-470; Sheldon et al., 1993, Clinical Chem. 39, 718-719; Fodor et al., 1991, Science 251, 767-773) and handbooks (DNA Microarrays: A Molecular Cloning Manual. Bowtell & Sambrook, eds. Cold Spring Harbor Laboratory Press (2002) ISBN: 0-87969-625-7).

A skilled person will be able to obtain arrays according to his own design and corresponding array reading equipment from specialized suppliers (for instance Affymetrix Corp., Santa Clara, Calif., USA for DNA arrays and Ciphergen Biosystems, Fremont, Calif., USA for ProteinChip Array).

In a preferred embodiment, a method according to the invention comprises a comparison between at least two environmental conditions, namely a standard condition and an experimental condition, as a result of which the possibly detected change in a composition of a microbial population can be ascribed to the changed environmental condition.

Preferably, these measurements on a composition of a population are quantitative, but semi-quantitative or qualitative measurements may also be used.

The determination of an environmental condition by measuring a composition of a microbial population exposed to this environmental condition can be carried out as a single measurement. Also, a composition of a microbial population can be measured upon exposure to a plurality of environmental conditions, with one particular, defined environmental parameter being set at different values. In this manner, specific changes in a composition of a microbial population resulting from this changing environmental parameter can be determined.

In measuring a composition of a microbial population exposed to a plurality of known environmental conditions with one particular, defined environmental parameter being set at different values, such as different pH, it is possible to obtain reference measurements on the basis of which a reference data file can be built up. Such a data file comprises, for instance, a first variable p of different values at which an environmental parameter has been set, and a second variable X of different compositions of a microbial population which has been exposed to this environmental parameter.

To measurements of a composition of a microbial population, different analysis techniques may be applied, such as a statistical analysis, for instance a multivariate analysis, or other analysis techniques as they currently exist or as they can be developed for use in an embodiment according to the invention. In a method according to the present invention, use of analysis methods such as “self-organizing maps”, hierarchical clustering, “multidimensional scaling”, principal component analysis, “supervised learning”, “k-nearest neighbors”, “support vector machines”, discriminant analysis and “partial least square” methods are preferred. The analysis of the collection of measurement results which together describe a change in a composition of a microbial population resulting from changing, defined environmental conditions, results in a value for the environmental parameter and thus for the environmental condition.

For determining an unknown environmental condition by means of a method according to the invention, it is preferred to carry out the reference measurements in a stage preceding the determination of the unknown environmental condition. A measured composition of a microbial population which has been exposed to the unknown environmental condition then yields a value for this environmental condition after correlation of this composition of a microbial population with the data file mentioned.

A reference data file as used in embodiments of the present invention may, for instance, be in the form of a virtual calibration line. In that case, a composition of a microbial population will, for instance, have been entered into the data file as a variable with a real numerical value and a parameter of an environmental condition will have been entered into the data file as another variable with a real numerical value.

Further, the dynamics of a changing composition of a microbial population can be stored as a result in a data file; in this manner, changes in an environmental condition can already be detected in a very early stage. Herein, dynamics is understood to mean the transition characteristic of a composition of a microbial population which is in the stage of transition from a first substantially stable condition to a second substantially stable condition. Such a transition characteristic can, for instance, comprise a brief or sudden increase of a marginally present organism, or a change in the mass proportion of a particular organism in the population as a result of the reduction of the cell size.

The use of a data file of compositions of a microbial population from a particular process may comprise, for instance, comparing the result of a measurement with results of previous measurements and, also, adding the new measurement to the data file as a new reference measurement. The data file will thus increase in size and detail so that results are provided with an increasingly solid basis of more and more new results.

The comparison of the result of a measurement on a composition of a microbial population with results of previous measurements can very suitably be carried out by statistical correlation of results. In an alternative embodiment, the determination of an environmental condition therefore comprises inter alia the step of correlating a composition of a microbial population with a previously compiled data file of a plurality of compositions obtained through exposure of the microbial population to a plurality of environmental conditions.

A method according to the invention can very suitably be used for inter alia:
quality control of water (mineral water, process water or purification water) by measuring shifts in microbial populations;
control (and optionally adjustment) of food preparation processes by measuring starter cultures (inter alia for cheese, dairy and meat industries);
optimization of crop cultivation in agriculture and horticulture by analysis of soil flora;
detection of the proliferation of undesired microorganisms such as decay organisms, pathogens and quality-reducing microorganisms in a product by exposing the product to a population of microorganisms;
detection of pollution in the (water) soil by analysis of (water) soil flora; and
monitoring of indigenous flora (the natural microbial population) of biologically cultivated products for control of quality and authenticity.

### EXPERIMENTAL SECTION

This experimental section involves two independent experiments (A and B) in which the influence of the physical, chemical environment on a microbial population is determined.

Experiment A relates to the environmental influence on an artificial population which was analyzed by means of both selective cultivation media and a molecular measuring device.

Experiment B relates to natural environmental influences on a bacterial population in commercially available mineral water packaged in bottles. Because water bacteria cannot be distinguished by means of selective cultivation media, cultivation is not possible and a molecular measuring device is eminently suitable for analysis of a deviant mineral water quality. The neutral nature of mineral water makes it very sensitive to sensory deviations. The deviations are of a chemical nature. A trained nose can quickly detect a chemical deviation. Untrained people are not always able to recognize a deviation in a low concentration. Chemical analysis is not sufficiently sensitive for detection of deviant mineral waters either. Therefore, chemical deviations can well be recognized via the analysis of bacterial populations by the molecular measuring device.

Materials and Methods

Microbiological Analysis

For the artificial population in experiment A, 6 bacterial strains were used: Listeria monocytogenes ATCC19114, Pseudomonas putida S12, Salmonella ser. typhymurium ATCC 13311, Lactobacillus plantarum ATCC 14917, Bacillus subtilis Bacl-12 NCCB 93057 and Lactobacillus casei ATCC 393. The strains were grown overnight in Brain Heart Infusion (BHI) broth (OXOID) at 37° C., 100 rpm. Of the overnight cultures, a mixture was made of 105 colony-forming units (CFU)/ml per strain. This mixture was used to graft 20 ml of BHI in 100-ml glass bottles to. Per volume of 20 ml, 200 μl of bacteria mixture was added.

To the mixture cultures, the following conditions were applied:

1. BHI pH 7, at 30° C.; reference population

2. BHIpH7, at 48° C.

3. BHI pH 7, with kanamycin 40 μg/ml at 30° C.

4. BHI pH 5.0, at 30° C.

5. BHI pH 7, with 1% toluene and 1 mM of MgCl2 at 30° C.

After t=0, 6, 24 and 48 hours, by means of a 1 μl-loop, the mixture cultures were plated onto the following selective nutrient media: MRSA (OXOID), MRSA (OXOID) met 40 ug/ml kanamycin, Brilliant Green Agar (BGA, OXOID), PALCAM (OXOID), Pseudomanas Agar Base (PAB, OXOID) and MYP (OXOID). The 6 bacterial strains were also individually streaked on the above nutrient media. Further, after 6, 24 and 48 hours, 6×1 ml of mixture culture was centrifuged (3 min, 10,000 g), after which the cell pellets were stored at −80° C. for DNA isolation.

Molecular Population Analysis

DNA isolation from the bacterial populations was carried out according to the BeadBeater method (Hurley et al., 1987 J. Clin. Microbiol. 25.2227-9 and erratum in J. Clin. Microbiol. 26:1077). For this purpose, the cell pellets were defrosted, after which 400 μl of TE, 400 μl of zirconium beads of 0.1 mm and 400 μl of phenol were added. This mixture was beaten for 2 min at medium setting. Then, it was centrifuged for 5 minutes in an Eppendorf centrifuge at 24,000 g. The upper phase was extracted using 500 μl of chloroform and centrifuged (24,000 g in Eppendorf centrifuge). The water phase was transferred to a new Eppendorf tube, after which the DNA was deposited by adding 1/10 volume of 3M of sodium acetate with pH 5.2, glycogen (200 μg/ml) and 2 volumes of 96% ethanol at −80° C. After 1 hour, this cold mixture was centrifuged in an Eppendorf centrifuge for 2 minutes.

The pellet was washed using 70% ethanol and vacuum-dried. Then, the DNA was dissolved in TE with 100 μg/ml RNase and was incubated for 30 minutes at 37° C.

To make the composition of the bacterial population visible, a denaturing gradient gel electrophoresis (DGGE) was used. For this purpose, the protocol of Favier et al. (2002) Appl Environ Microbiol. 2002 68:219-26 was used. In essence, 5 to 40 ng of DNA of the bacterial mixtures were brought into a PCR reaction, in which the V6 to V8 regions of the 16S rDNA were amplified.

For the DGGE, the PCR was started with the following composition:

1 μl or 10 μl of DNA from stock

5 μl of Goldstar buffer HOT (Eurogentec, Seraing, Belgium)

5 μl of d'NTPs

6 μl of Goldstar MgCl2 (25 mM)

1 μl of U968-GC (10 pmol/μl)

1 μl of L1401 (10 pmol/μl)

0.25 μl of Goldstar taq polymerase HOT

30.75 or 21.75 μl milli Q

The following PCR program was used for amplification: 10 min 94° C.; 30× (10 sec 94° C., 20 sec 56° C., and 40 sec 68° C.); 7 min 68° C.; 4° C. hold. To check the PCR, 10 μl of PCR product was electrophoresed in a 1.2% agarose gel.

Results

Experiment A: Influence of Artificial Conditions on a Bacterial Population.

Bacterial Growth

The development of the change in the bacterial population was followed by streaking on selective nutrient media. This provided a rough indication of the changes occurring in the populations as a result of the prevailing chemical physical conditions. In BHI medium at 30° C., all introduced species were still found. At 48° C., B. subtilis became dominant within a very short time and all other species were no longer found on the selective media. In BHI medium with kanamycin, L. plantarum became dominant. Initially, the other species were still found at t=6 hours. After this, only L. plantarum and P. putida were present. In BHI medium at pH 4.5, the Lactobacillus species in particular became dominant. Here as well, we initially also find the other species on the selective media at t=6 hours. On BHI medium with toluene, we find a rapid dying of all species. At t=48, growth of L. plantarum is detected again.

Molecular Composition of Bacterial Population

The development of the change in the bacterial population was also followed by means of DGGE analysis of the ribosomal nucleotide sequences present. Here, the bands represent individual species.

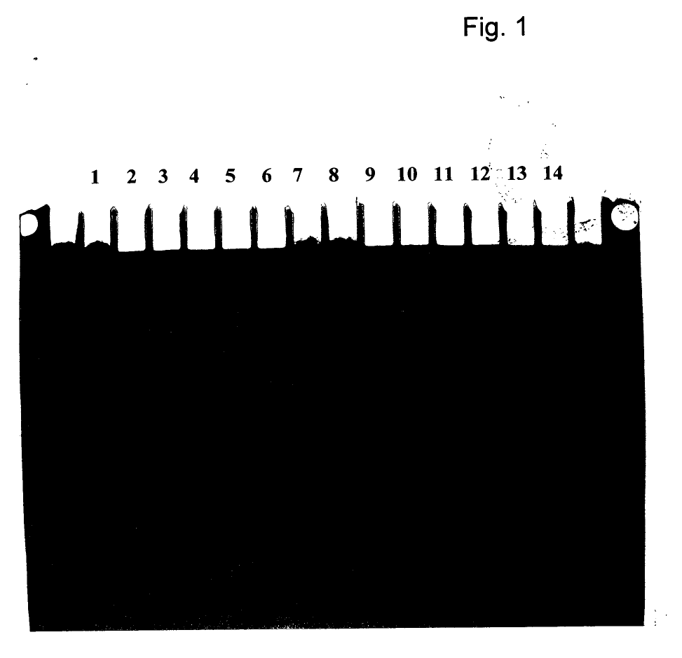

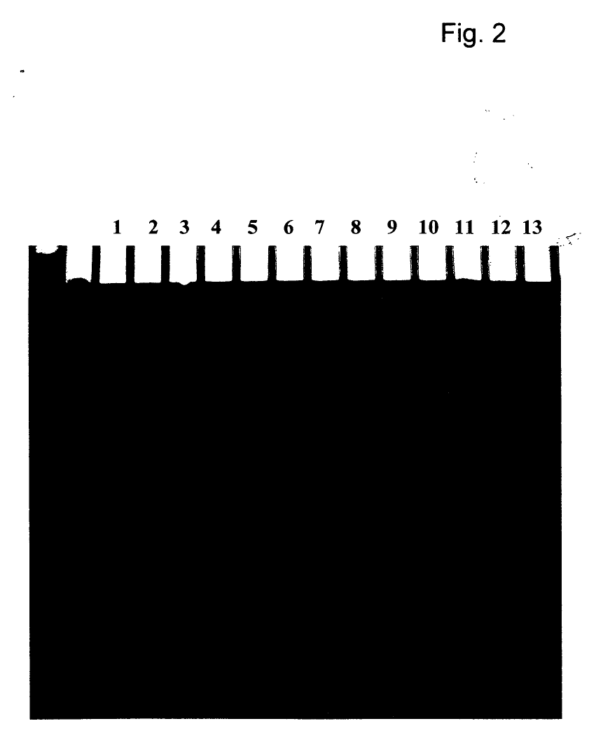

FIGS. 1 and 2 show the DGGE band patterns as a result of time and environmental condition in the different media.

FIG. 1 shows the effect of process conditions on the bacterial population consisting of Li. monocytogenes, P. putida, Salmonella ser. typhymurium, L. plantarum, B. subtilis, and L. casei after 6 hours.

Lane 1, reference population; lane 2, Li. monocytogenes; lane 3, P. putida; lane 4, S. typhymurium; lane 5, L. plantarum; lane 6, B. subtilis; lane 7, L. casei; lane 8, reference population t=0 hours; lane 9, reference population t=6 hours; lane 10, 48° C., t=6 hours; lane 11, kanamycin t=6 hours; lane 12, pH 4.5, t=6 hours; lane 13, toluene t=6 hours; and lane 14, reference population.

FIG. 2 shows the effect of process conditions on the bacterial population consisting of Li. monocytogenes, P. putida, Salmonella ser. typhymurium, B. subtilis, L. casei and L. plantarum after 24 and 48 hours. Lane 1, reference population t=0; lane 2, reference population t=24 hours; lane 3, 48° C.; lane 4, kanamycin t=24 hours; lane 5, pH 4.5, t=24 hours; lane 6, toluene t=24 hours; lane 7, reference population t=0; lane 8, reference population t=48 hours; lane 9, 48° C., t=48 hours; lane 10, kanamycin t=48 hours; lane 11, pH 4.5, t=48 hours; lane 12, toluene t=48 hours; and lane 13, reference population t=0.

Parallel to the picture shown by analysis of the bacterial growth on the selective media, we see that the population shifts in time. In the reference population in BHI at 30° C., all bands remained visible at different times of sampling. With use of other physical chemical conditions, changes of the band pattern were detected. At t=6 hours, the change is often not clearly visible yet, with the exception of the incubation at 48° C., where the B. subtilis band became very dominant (FIG. 1, lane 10). Great differences compared to the t=0 situation occurred at t=24 and 48 hours. These differences run parallel to what was visible on the selective nutrient media. The exception are the reference populations at t=24 and 48 hours (FIG. 2, lanes 2 and 8, respectively). In these populations, Salmonella and P. putida, in particular, become dominant. In the presence of kanamycin, at. t=24 hours, L. plantarum is found as a dominant species with a small proportion of P. putida. Then, at t=48 hours, P. putida was found to have become dominant with a limited proportion of L. plantarum. At low pH (4.5), L. plantarum in particular became dominant. L. casei plays a minor role at low pH. In the presence of toluene, a strong decline of all species can be seen until t=24, with only light bands being visible. Then, at t=48, L. plantarum was found to have become dominant. The latter is surprising, since L. plantarum had not been described as a toluene-tolerant microorganism. In conclusion, it can be stated that the physical chemical condition determines the composition of the bacterial population. Depending on the condition, a different population composition arises.

Experiment B: Influence of Natural Condition on a Bacterial Population.

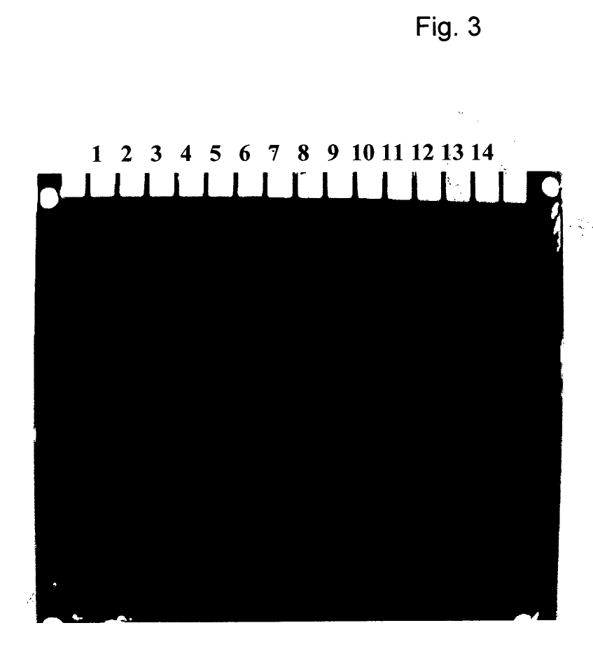

Experiment B relates to natural environmental influences on a bacterial population in commercially available mineral water packaged in bottles. The results are shown in FIG. 3. FIG. 3 shows DGGE patterns of mineral water samples from one supplier. Lanes 1 to 7 are the “baseline” population of good mineral water without sensory deviations. Lanes 8 to 14 represent the patterns of mineral water with a sensory deviation.

The baseline DGGE patterns of qualitatively good mineral water are all very similar (FIG. 3, lanes 2 to 7). The corresponding mineral waters did not show any sensory defect. The DGGE patterns of the sensorially deviant mineral waters showed deviant patterns compared to the baseline patterns. Sample (FIG. 3, lane 13) in particular deviates very strongly. This was also the sample with the strongest sensory deviation. In this water, one species had become so dominant that all other bands in the DGGE patterns had disappeared. The sensory changes which had occurred could only be detected by smelling by a trained inspector. Untrained noses could often not detect the sensory defect. Neither could changes be measured yet by state of the art methodologies, via chemical analysis, since they occur at ppm level. Analysis of the bacterial population did show clear differences (FIG. 3, lanes 8 to 14).

In conclusion, we may state that subtle differences in the chemical composition of mineral water can be made clearly visible by the molecular measuring device directed to the bacterial population.

### CONCLUSION

Physical chemical changes in an environment can be measured and read out on the basis of a change in the composition of a bacterial population which can be measured by a measuring device based on molecular principles.

1. A method for determining an environmental condition by measuring a composition of a microbial population which has been exposed to said environmental condition.

2. A method for determining changes in an environmental condition by measuring changes in a composition of a microbial population which has been exposed to said changes in an environmental condition.

3. A method for determining an environmental condition, comprising measuring a composition of a microbial population which has been exposed to said environmental condition, correlating said composition to a previously compiled reference data file of a plurality of compositions obtained through exposure of said microbial population to a plurality of environmental conditions and determining said environmental condition on the basis of the outcome of said correlation.

4. A method according to claim 1, wherein said microbial population comprises bacteria, fungi and/or yeasts.

5. A method according to claims 4, 14 and 15, wherein said microbial population is intestinal flora or soil flora.

6. A method according to claims 4, 14 and 15, wherein said microbial population is a microbial population introduced into or occurring naturally in a specific process.

7. A method according to claim 1, wherein said measurement comprises the use of taxon-specific markers.

8. A method according to claim 7, wherein said taxon-specific markers are nucleic acid markers.

9. A method according to claim 7, wherein said composition of a microbial population is determined by means of one or more microarrays.

10. A method for controlling or monitoring an environmental condition, comprising a method according to claim 1.

11. A method for controlling a process, comprising a method according to claim 10.

12. Use of a method according to claim 1, for quality control of water, for control of a food preparation process, for optimization of crop cultivation, for the optimization of biodegradation in the soil, for the detection of soil pollution or for the detection of undesired microorganisms.

13. Use of a method according to claim 1, for determining a chemical substance in the soil, the air and/or in aqueous environmental.

14. A method according to claim 2, wherein said microbial population comprises bacteria, fungi and/or yeasts.

15. A method according to claim 3, wherein said microbial population comprises bacteria, fungi and/or yeasts.

16. A method according to claim 4, wherein said measurement comprises the use of taxon-specific markers.

17. A method according to claim 14, wherein said measurement comprises the use of taxon-specific markers.

18. A method according to claim 15, wherein said measurement comprises the use of taxon-specific markers.

19. A method according to claim 17, wherein said taxon-specific markers are nucleic acid markers.

20. A method according to claim 18, wherein said taxon-specific markers are nucleic acid markers.

21. A method according to claim 8, wherein said composition of a microbial population is determined by means of one or more microarrays.

22. A method according to claim 17, wherein said composition of a microbial population is determined by means of one or more microarrays.

23. A method according to claim 18, wherein said composition of a microbial population is determined by means of one or more microarrays.

24. A method according to claim 19, wherein said composition of a microbial population is determined by means of one or more microarrays.

25. A method according to claim 20, wherein said composition of a microbial population is determined by means of one or more microarrays.

26. A method for controlling or monitoring an environmental condition, comprising a method according to claim 2.

27. A method for controlling or monitoring an environmental condition, comprising a method according to claim 3.

28. A method for controlling a process, comprising a method according to claim 26.

29. A method for controlling a process, comprising a method according to claim 27.

30. Use of a method according to claim 2, for quality control of water, for control of a food preparation process, for optimization of crop cultivation, for the optimization of biodegradation in the soil, for the detection of soil pollution or for the detection of undesired microorganisms.

31. Use of a method according to claim 3, for quality control of water, for control of a food preparation process, for optimization of crop cultivation, for the optimization of biodegradation in the soil, for the detection of soil pollution or for the detection of undesired microorganisms.

32. Use of a method according to claim 2, for determining a chemical substance in the soil, the air and/or in aqueous environmental.

33. Use of a method according to claim 3, for determining a chemical substance in the soil, the air and/or in aqueous environmental.
